(19) United States
(12) Patent Application Publication
(10) Pub. No.: US 2011/0142955 A1
(43) Pub. Date: Jun. 16, 2011

(54) AGENT FOR AMELIORATING STRESS-INDUCED IMMUNE FUNCTION MODULATION

(75) Inventors: Yoshiya Sato, Nakagami-gun (JP); Kaiissar Mannoor, Nakagami-gun (JP); Hisami Watanabe, Nakagami-gun (JP); Kikuji Yamaguchi, Atami-Shi (JP); Mayu Tsukamoto, Tokyo (JP)

(73) Assignees: Kikuji YAMAGUCHI, Atami-Shi, Shizuoka (JP); UNIVERSITY OF THE RYUKYUS, Nakagami-gun, Okinawa (JP)

(21) Appl. No.: 13/054,367

(22) PCT Filed: Jul. 15, 2009
(86) PCT No.: PCT/JP2009/062775

§ 371 Date: Mar. 1, 2011

(30) Foreign Application Priority Data
Jul. 16, 2008 (JP) 2008-184720

(51) Int. Cl. A61K 35/64; A61P 37/02

(52) U.S. Cl. 424/537

(57) ABSTRACT

The present invention relates to an agent for ameliorating stress-induced immune function modulation which comprises royal jelly. In addition, the present invention provides an anti-stress agent, pharmaceutical, food or beverage, or food additive that contains royal jelly as an active ingredient thereof and prevents or alleviates various somatic symptoms caused by stress.

### TECHNICAL FIELD

The present invention relates to an anti-stress agent comprising royal jelly, and more specifically, to an agent for ameliorating stress-induced immune function modulation. In addition, the present invention relates to a pharmaceutical, food or beverage, or food additive which comprises royal jelly as an ingredient thereof and prevents or alleviates various somatic symptoms caused by stress.

Prolonged, intense stress has been firmly established to have a detrimental effect on health, and stress is intimately involved with the onset of cardiovascular, gastrointestinal and mental diseases. In addition, stress is also known to cause various disorders of the immune system.

However, the effect of royal jelly on stress has yet to be determined in detail.

### DISCLOSURE OF THE INVENTION

### Problems to be Solved by the Invention

As a result of conducting extensive research, the inventors of the present invention determined for the first time that royal jelly regulates immune functions, ameliorates modulation of immune functions induced by stress, and has an effective action in terms of protecting the body against stress, thereby leading to completion of the present invention.

The present invention provides an anti-stress agent and an agent for ameliorating stress-induced immune function modulation. In addition, the present invention provides a pharmaceutical, food or beverage, or food additive that prevents or alleviates various somatic symptoms caused by stress.

The present invention relates to the following:

1. an agent for ameliorating stress-induced immune function modulation which comprises royal jelly;

2. a pharmaceutical having an ameliorative action on stress-induced immune function modulation, which comprises royal jelly as an active ingredient thereof;

3. a food or beverage having an ameliorative action on stress-induced immune function modulation, which comprises royal jelly as an active ingredient thereof;

4. a food additive having an ameliorative action on stress-induced immune function modulation, which comprises royal jelly as an active ingredient thereof;

5. a method for ameliorating stress-induced immune function modulation, which comprises ingesting royal jelly;

6. the agent for ameliorating stress-induced immune function modulation according to the above 1, which has an action that restores decreases in peripheral lymphocytes induced by stress;

7. the agent for ameliorating stress-induced immune function modulation according to the above 1, which has an action that restores increases in granular leukocytes induced by stress;

8. the agent for ameliorating stress-induced immune function modulation according to the above 1, which has an action that restores thymus atrophy induced by stress; and,

9. an anti-stress agent comprising royal jelly.

### BRIEF DESCRIPTION OF THE DRAWINGS

FIG. 1 shows graphs indicating the dynamics of absolute numbers of peripheral leukocytes (WBC), lymphocytes and granulocytes in C57BL/6 mice following oral administration of royal jelly and PBS. Graph A indicates absolute WBC count, graph B indicates absolute lymphocyte count, and graph C indicates absolute granulocyte count. Royal jelly was diluted with PBS, and 30 μl of the diluted royal jelly solution containing 2.0 mg of protein were administered to each mouse using a regimen consisting of administering once every two days over the course of 6 weeks.

FIG. 2 shows graphs indicating the dynamics of the absolute numbers of T (CD3⁺) and B (B220⁺) lymphocytes in the peripheral blood of C57BL/6 mice following oral administration of royal jelly and PBS. Graph A indicates the absolute T lymphocyte cell count, while graph B indicates the absolute B lymphocyte count.

FIG. 3 shows a graph indicating the absolute numbers of mononuclear cells (MNC) in the liver, spleen and thymus of C57BL/6 mice on day 42 following oral administration of royal jelly.

FIG. 4 shows graphs indicating the absolute numbers of CD3⁺ T cells, B220⁺ B cells, CD4⁺ T cells and CD8⁺ T cells in the liver of mice on day 42 following oral administration of royal jelly.

FIG. 5 shows a graph indicating the absolute numbers of CD4⁺ T cells and CD8⁺ T cells in the spleen of C57BL/6 mice on day 42 following oral administration of royal jelly.

FIG. 6 shows graphs indicating the absolute numbers of peripheral leukocytes (WBC), lymphocytes and granulocytes in stress-loaded C57BL/6 mice. Graph A indicates absolute WBC count, graph B indicates absolute lymphocyte count, and graph C indicates absolute granulocyte count. Royal jelly was administered to mice subjected to restraint stress for 12 hours twice a week using the dosing regimen described in FIG. 1. PBS not containing royal jelly was administered as a control. A P value of less than 0.05 was evaluated as constituting statistical significance.

FIG. 7 shows graphs indicating the absolute numbers of CD3⁺ T cells and B220⁺ B cells in the peripheral blood of mice following oral administration of royal jelly and PBS supplemented with the restraint stress described in FIG. 6. Graph A indicates absolute T lymphocyte count, while graph B indicates absolute B lymphocyte count. A P value of less than 0.05 was evaluated as constituting statistical significance.

FIG. 8 is a graph indicating the absolute numbers of mononuclear cells (MNC) in the liver, spleen and thymus of C57BL/6 mice on day 42 following oral administration of royal jelly.

FIG. 9 shows graphs indicating the absolute numbers of CD4⁺ T cells, CD8⁺ T cells and CD4⁺/CD8⁺ double-positive T cells in the thymus of C57BL/6 mice on day 42 following oral administration of royal jelly supplemented with restraint stress twice a week.

### BEST MODE FOR CARRYING OUT THE INVENTION

Royal jelly is a milky, yellowish-white, jelly-like liquid that is secreted from pharyngeal glands and mandibular glands present in the head segment of worker honeybees following consumption of pollen by these bees for mainly 3 to 10 days after emergence and the metabolism of that pollen in an organ known as the cardiac tube. In a honeybee society, royal jelly is given as a special food to the queen bee. After having ingested the royal jelly, the queen bee grows to twice the size of other worker bees and is able to maintain a long life span of 3 to 5 years as compared with the average life span of worker bees of 35 to 40 days. During this time, the queen bee lays 2,000 to 3,000 eggs per day, thereby maintaining the sophisticated social order of honeybees.

Restraint stress is well known to inhibit the response of T and B lymphocytes in peripheral blood while conversely enhancing the response of granular leukocytes. On the other hand, since royal jelly produced by honeybees is said to have an anti-stress effect and an effect that facilitates recovery from fatigue, the inventors of the present invention attempted to examine whether or not abnormal changes in the counts of lymphocytes and granular leukocytes induced by stress are restored by royal jelly by investigating the dynamics of these cells in the peripheral blood and organs related to the immune system (thymus, spleen and liver).

In the case of having administered royal jelly to mice not subjected to stress, total leukocyte and lymphocyte counts in the peripheral blood and individual organs did not demonstrate any particularly remarkable changes, including changes in the counts of T and B lymphocytes and the counts of CD4/CD8 T lymphocytes subsets, and there were also no changes observed in the count of granular leukocytes in peripheral blood.

On the other hand, a remarkable decrease in all lymphocytes, including T and B lymphocytes in peripheral blood, and conversely an increase in the count of granular leukocytes, were observed in mice subjected to restraint stress in the case of not administering royal jelly to the mice. In addition, a decreasing trend in mononuclear cells (MNC) counts was demonstrated in the spleen and thymus of mice subjected to stress loading.

When royal jelly was administered to these mice subjected to restraint stress, abnormalities in peripheral lymphocyte and granular leukocyte counts tended to recover considerably. However, these recovery effects were limited to days 14 and 28 after stress loading, while recovery effects were unable to be observed on day 42. In addition, administration of royal jelly demonstrated the effect of restoring thymus cell counts, including CD4⁺ and CD8⁺ positive cells, even though that restoration was not adequate, in the thymus that had undergone considerable atrophy due to restraint stress. Recovery of lymphocyte counts in the peripheral blood and thymus was demonstrated to be achieved in a favorable balance among T/B lymphocyte, CD4/CD8 T lymphocyte and double-positive CD4⁺/CD8⁺ lymphocyte subsets.

On the basis of these results, royal jelly was clearly demonstrated to have an anti-stress action that restores immune function modulation induced by stress in the proper balance without providing a remarkable impact on immune function in the normal state.

Prolonged, intense stress has been firmly established to have a detrimental effect on health, and stress is intimately involved with the onset of cardiovascular, gastrointestinal and mental diseases. In addition, stress is also known to cause various disorders of the immune system (1-3).

In addition to prolonged stress being known to cause disruption of both cellular immunity and humoral immunity (4), stress has also been reported to cause immunodeficiency, including prominent atrophy of the thymus, inhibition of lymphogenesis, and decreased production of antibodies and cytokines, by inhibiting by being mediated by adrenocortical hormones (5-7).

In addition, in mice subjected to restraint stress, stress has been reported to cause lymphocytopenia and conversely granulocytosis, thymus atrophy and numerous other modulations relating to immune functions (8, 9).

Moreover, restraint stress is also thought to not only induce apoptosis of thymus cells to lead to a reduction in CD4/CD8 double-positive cells in the thymus, but also result in a remarkable decrease in peripheral lymphocytes accompanying apoptosis of mature lymphocytes (6, 7).

A direct relationship has been observed between this thymus atrophy induced by stress and increased glucocorticoid levels (6, 7, 10).

On the other hand, royal jelly is a milky white, jelly-like liquid for promoting growth and development of the queen bee that is secreted from the pharyngeal glands of worker bees. Larva that have been fed royal jelly are able to develop biological properties superior to those of worker bees in terms of body size, vitality, stamina and life span. For example, in contrast to worker bees having a life span of 35 to 40 days, the life span of a queen bee can reach 5 to 7 years. In addition, royal jelly is also known to demonstrate a diverse range of functions in humans as well (11-14), and one of those functions involves an effect on immune function. Royal jelly has been reported to be involved in regulation of immune function by promoting antibody production and growth of immunocompetent cells in mice (15-17).

Therefore, the inventors of the present invention conducted the present experiment for the purpose of examining the degree to which stress-induced impairment of immune function is restored by royal jelly. In this experiment, royal jelly was clearly determined to demonstrate an effect of returning stress-induced abnormal peripheral lymphocyte and granular leukocyte counts to normal as well as restore atrophied thymus following oral administration of royal jelly to mice subjected to restraint stress. This result clearly indicated for the first time that royal jelly regulates immune function and has an effective action in terms of protecting the body against stress.

A conventionally known, any royal jelly can be used in the present invention. Examples of species of honeybees that secrete the royal jelly of the present invention include Apis mellifera, Apis cerana, Apis dorsata and Apis florea.

Examples of production sites of the royal jelly of the present invention include Japan, South America, North America, Australia, China and Europe. This royal jelly can be advantageously used, regardless of the form, purity or preparation method thereof, provided it demonstrates the effects on treatment and prevention of diseases related to production of autoantibodies when applied to mammals, including humans, either in an unprocessed form or after having been treated with a suitable purification process.

As is commonly known, royal jelly has already long been widely used as a health maintenance food or food for specified health uses, and its safety has been adequately verified.

The composition of the present invention can incorporate components permitted for oral, transcutaneous or external skin use in mammals, including humans, in addition to royal jelly serving as the active ingredient thereof. Examples of such components include water, alcohol, starch, proteins, amino acids, fiber, sugars, lipids, fatty acids, vitamins, minerals, flavorings, colorants, sweeteners, seasonings, spices, antiseptics, emulsifiers, surfactants, vehicles, extenders, thickeners and preservatives. The composition of the present invention can advantageously contain one type or two or more types of these components.

The composition of the present invention can be used for a conventionally known arbitrary administration route, such as orally or parenterally. The effective amount of intake or dose of the composition of the present invention can be suitably determined according to type, age or gender and so forth of the target mammal, including humans, and for example, may be ingested or administered orally normally at 0.01 to 100 mg/administration, and preferably 0.1 to 50 mg/administration, in mass of the active ingredient, per kg of body weight either once a day or divided among several administrations per day, or daily or at intervals of 1 day or more corresponding to its effect.

In producing the composition of the present invention, royal jelly is mixed at a suitable blending ratio with one type or two or more types of components able to be used in fields such as foods, beverages, cosmetics, pharmaceuticals, quasi drugs, feeds, animal diets or pet foods, followed by suitably undergoing such processes as dilution, concentration, drying, filtration or centrifugal separation, to form into a desired form to prepare a composition incorporating an antiallergic agent, in view of target animals and ingestion methods or administration methods. There are no particular limitations on the order in which each component is incorporated and the times at which each process is carried out provided they do not impair the effects of the present invention.

The composition of the present invention can be used, for example, in the form of a food or beverage such as a fermented milk drink or lactic acid bacteria drink. In addition, it can also be used in the form of a pharmaceutical such as tablets.

### Example 1

The following provides a more detailed explanation of the present invention by indicating specific examples thereof.

Materials and Methods

Experimental Animals: Female C57BL/6 (B6) mice age 6 to 12 weeks were used in the experiments. The mice were housed in a specific pathogen free (SPF) environment at the Institute for Animal Experiments of the University of the Ryukyus throughout the duration of the experiments. In addition, all experiments were carried out according to an experiment protocol with permission based on the animal experiment guidelines of the University of the Ryukyus.

Royal Jelly and Administration Method: The royal jelly used in the experiments consisted of crude royal jelly produced by organic beekeeping according to the method of Kikuji Yamaguchi (18) and supplied from Japan Royal Jelly Co., Ltd. The royal jelly was diluted with PBS and 30 ml thereof (equivalent to 2 mg of protein) were orally administered to mice every two days. Mice administered PBS only according to the same schedule were used as a control.

Restraint Stress: The mice were stress-loaded by restraining the mice between pieces of stainless steel mesh so that they were unable to move freely. Restraint stress loading was carried out twice a week for 12 hours overnight for 6 consecutive weeks (19-21).

Analysis of Peripheral Blood Cells: Blood samples were collected from the mice making an incision in the tails of the mice and collecting the blood from the incision in heparinized capillary tubes. The blood samples were measured for the counts of lymphocytes, lymphocyte subsets and granulocytes. The collected blood was applied to a slide glass and stained with Giemsa stain followed by microscopically measuring the counts of lymphocytes and granular leukocytes based on the morphological characteristics of mononuclear cells (MNC). In addition, MNC were immunostained with fluorescence-labeled monoclonal antibodies followed by analysis of lymphocyte subsets based on the phenotype of the cell surfaces thereof (cell surface antigen type) using a flow cytometer.

Collection of Cells from Organs: The mice were sacrificed to be exsanguinated by cardiocentesis on day 42 after the start of administration of royal jelly followed by excision of the thymus, liver and spleen. MNC were collected from the thymus by slicing thymus tissue into thin sections in Eagle's MEM medium (containing 50 mM HUES) and passing through a 200 gauge stainless steel mesh. In the case of cells from the spleen, cells obtained by pressing the excised spleen through a 200 gauge stainless steel mesh were collected in MEM medium (containing 50 mM HEPES and 2% inactivated fetal bovine serum). The cell suspensions were subsequently centrifuged at 1500 rpm, the resulting sediment was treated for 3 minutes at 4° C. with 3 ml of erythrocyte lysis solution (155 mU of NH₄Cl, 10 mM KHCO₃, 170 mM Tris) to hemolyze the erythrocytes. Finally, MNC were washed by centrifugation and used after suspending in MEM medium. On the other hand, MNC were collected from the liver by similarly slicing the excised livers into thin sections and passing through a stainless steel mesh followed by suspending the resulting MNC in MEM medium, washing, layering over 35% Percoll solution and centrifuging for 15 minutes at 2000 rpm. Cells obtained by hemolyzing the resulting sediment for 10 minutes at 4° C. with 5.0 ml of erythrocyte lysis solution were used after washing two times by centrifugation (22). The resulting MNC were used in cell analyses after immunofluorescence staining using fluorescence-labeled monoclonal antibodies.

Flow Cytometry: Analyses using a flow cytometer were carried out using fluorescence-labeled monoclonal antibodies to mouse cell surface antigen. The monoclonal antibodies used (anti-CD8, anti-CD3, anti-CD4 and anti-CD45/B220 antibodies) were purchased as FITC-labeled antibodies from Pharmingen. In addition, PE-labeled anti-CD45 (clone 2D1) antibody purchased from Becton Dickinson was also used. When staining the labeled monoclonal antibodies, the cells were pretreated with anti-CD32/CD16 non-labeled monoclonal antibody solution to avoid non-specific staining followed by immunostaining using each labeled monoclonal antibody. Analysis of peripheral blood lymphocytes was carried out on CD45-positive cells gated with anti-CD45 labeled antibody. Non-viable cells in the cell suspensions were excluded from analysis by forward scatter, side scatter and propidium iodide gates.

Statistical Processing: The presence of a statistical difference was tested using the Student's t-test by computer software, and a P value of 0.05 or less was judged to constitute statistical significance.

Results
Effects of Royal Jelly on Peripheral Blood Total Leukocyte Count, Lymphocyte Count, Granular Leukocyte Count, and TIB Lymphocyte Count

In order to first investigate the effects of administration of royal jelly on immune cells in mice not subjected to stress, total leukocyte (WBC) counts, lymphocyte counts and granulocyte counts were compared in mouse groups administered royal jelly or PBS. As shown in FIGS. 1A to 1C, there were no large differences observed in peripheral blood WBC counts, lymphocyte counts or granulocyte counts between royal jelly-dosed mice and control mice dosed with PBS.

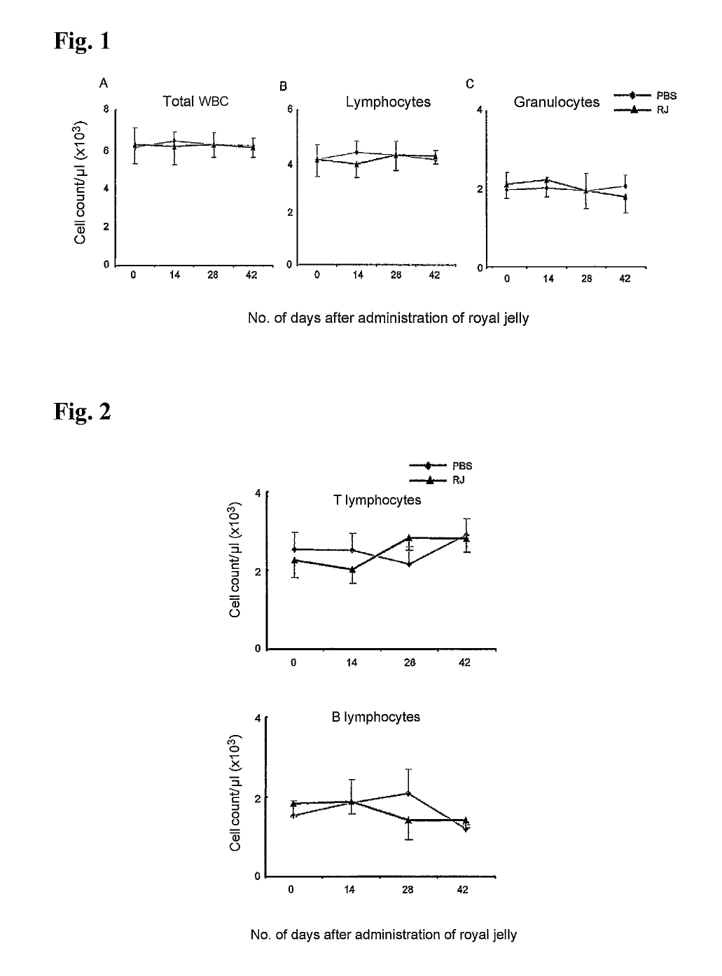

Moreover, whether or not administration of royal jelly also has an effect on T and B lymphocytes counts was also examined. As a result, there were no significant differences able to be observed attributable to administration of royal jelly between T and B lymphocytes counts (FIG. 2).
Effect of Administration of Royal Jelly on Leukocyte Counts and Lymphocyte Subsets in the Thymus, Liver and Spleen

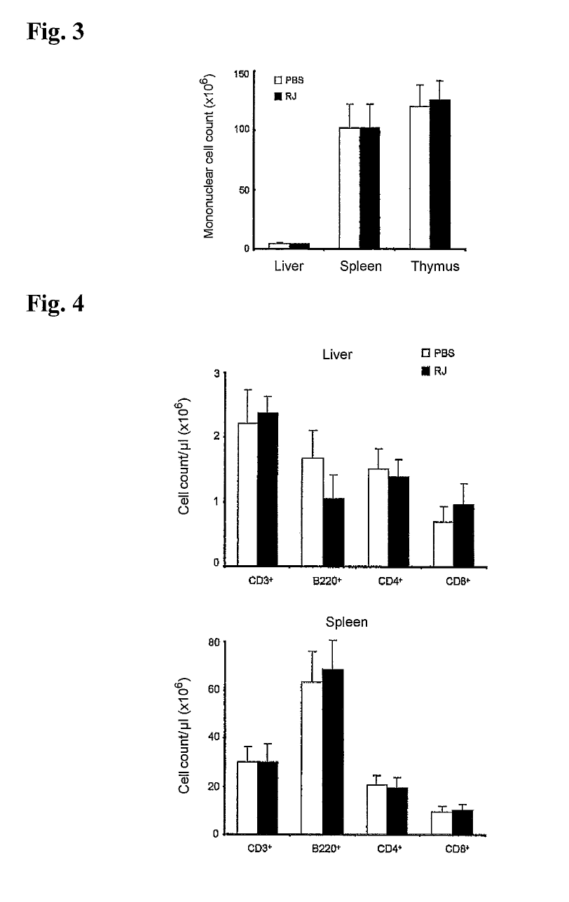

In order to examine whether or not oral administration of royal jelly has an effect on the dynamics of MNC in the thymus, liver and spleen in mice not subjected to stress loading, the total counts of MNC in the liver, spleen and thymus excised on day 42 after the start of the experiment were measured, and those results are shown in FIG. 3.

There were no differences whatsoever observed between royal jelly-dosed groups and PBS-dosed control groups in the counts of MNC distributed in these organs.

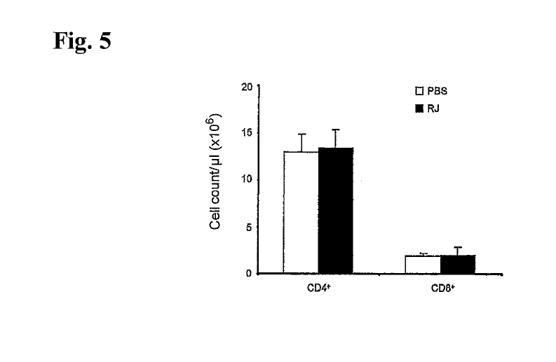

In addition, the counts of T lymphocytes (GD3⁺), B lymphocytes (B220⁺) and CD4⁺/CD8⁺ T lymphocytes subsets in the liver and spleen were compared for these MNC. As shown in FIG. 4, although a decreasing trend was observed for the count of B220⁺ cells in the liver, the difference was not statistically significant, and there were hardly any differences observed in MNC subsets in these organs. Although the effect of administration of royal jelly on CD4⁺ and CD8⁺ T cell subsets was also examined for thymus cells, hardly any differences in these subsets attributable to administration of royal jelly were able to be observed (FIG. 5).
Changes in Total Leukocyte, Lymphocyte and Granulocyte Counts in the Peripheral Blood of Restraint Stress Mice and Effect of Administration of Royal Jelly

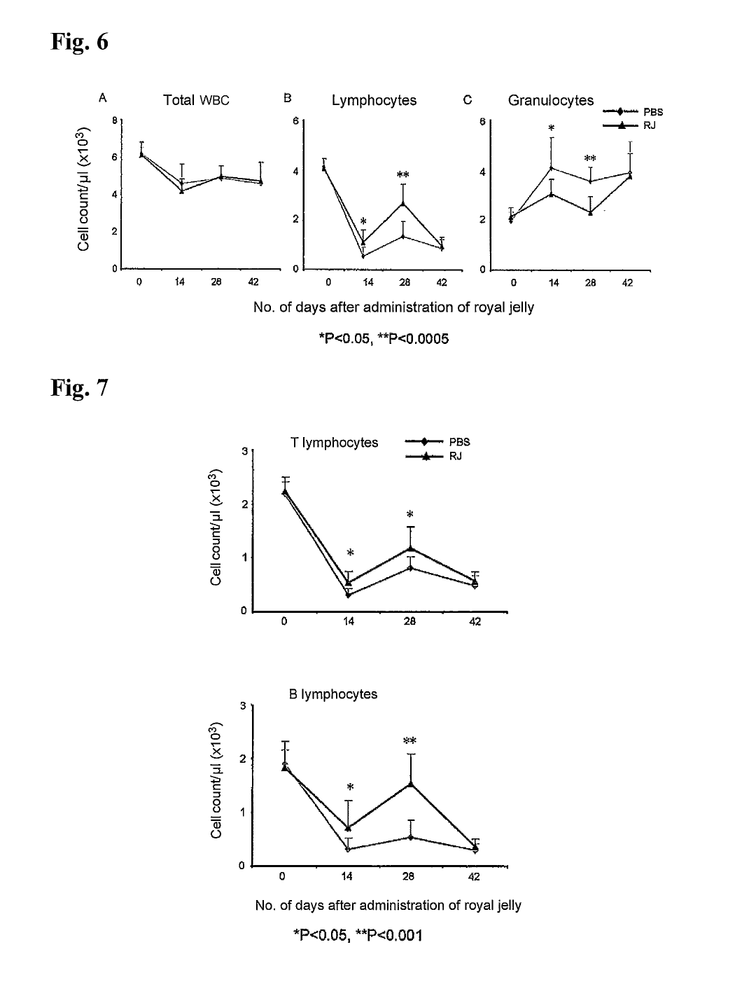

Peripheral blood WBC count and lymphocyte count demonstrated remarkable decreasing trends in the case of subjecting the mice to restraint stress (FIGS. 6A and 6B), while conversely the counts of granular leukocytes in peripheral blood demonstrated a rapid increasing trend following stress loading (FIG. 6C).

On the other hand, as a result of administering royal jelly to mouse subjected to stress, lymphocyte counts demonstrated significant increases in comparison with control mice on days 14 and 28 after the start of administration of royal jelly (day 14: p<0.05, day 28: p<0.0005) (FIG. 6B). Conversely, granular leukocyte counts that increased due to stress demonstrated remarkable decreases in comparison with the control mice on days 14 and 28 after the start of administration of royal jelly (FIG. 6C).

In reflection of these increases in lymphocyte counts and decreases in granular leukocyte counts in mice administered royal jelly after stress loading, there were no remarkable differences in peripheral blood WBC counts between the royal jelly dose group and PBS dose group (FIG. 6A).

In reflection of the decreases in lymphocyte counts and increases in granular leukocyte counts in mice subjected to stress, the ratio of granular leukocytes to lymphocytes was 7:1 on day 14 and 3:1 on day 28, while as a result of administering royal jelly, this ratio was 2.5:1 on day 14 and 1:1.2 on day 28, thus demonstrating nearly normal ratios. However, restorative effects induced by royal jelly were not observed as a result of continuing to subject the mice to stress, and there were no longer any differences observed between the royal jelly dose groups and PBS dose groups on day 42. These results suggest that, although royal jelly effectively maintains changes in immune cell counts induced by stress in a normal state in the short term, when stress is applied over an extended period of time, restorative effects are no longer adequately demonstrated.
Effect of Royal Jelly on Peripheral T and B Lymphocyte Counts in Restraint Stress Mice

Although royal jelly demonstrated the effect of restoring decreases in peripheral lymphocyte counts induced by stress, the question remained as to whether royal jelly demonstrates a restorative effect on T lymphocytes or B lymphocytes in peripheral blood. Therefore, when peripheral T lymphocyte and B lymphocyte counts were examined between a royal jelly dose group and a PBS control group, counts of both of these lymphocytes were observed to similarly recover on days 14 and 28 on which lymphocyte counts demonstrated remarkable recovery (FIGS. 7A and 7B). As a result, there were confirmed to be no large deviations in the ratio of T to B lymphocytes within the times total lymphocyte counts demonstrate a recovering trend.
Dynamics of Leukocytes in Thymus, Liver and Spleen of Restraint Stress Mice and Effect of Royal Jelly

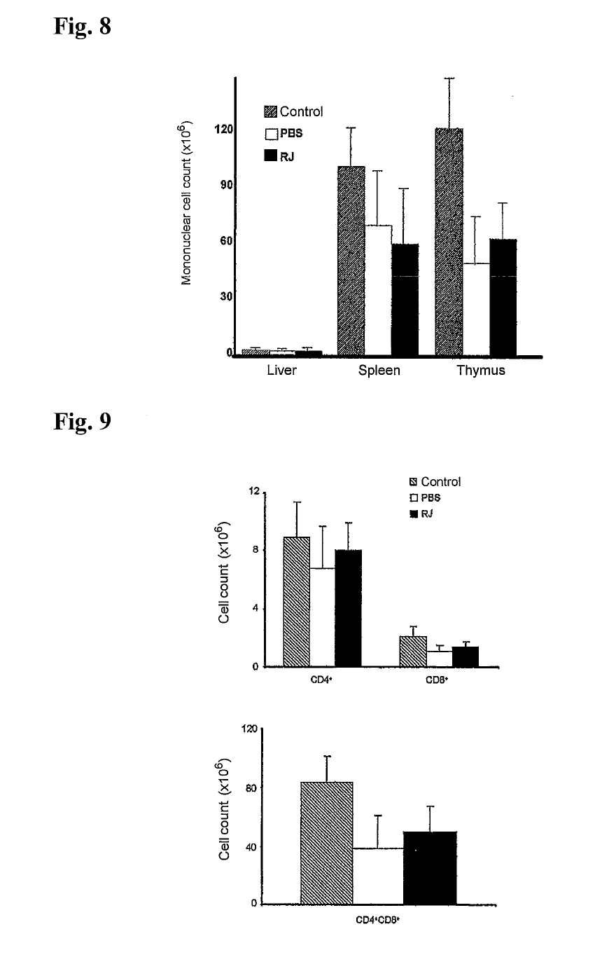

Cell counts in the thymus, liver and spleen on day 42 in stressed mice were compared in order to observe changes in MNC counts in the thymus, liver and spleen of mice subjected to restraint stress and restorative effects induced by administration of royal jelly. Cell counts in the spleen and thymus demonstrated remarkable decreases as a result of being subjected to restraint stress, and although restorative effects on cells in the spleen were not observed to be induced by administration of royal jelly to these mice, cell counts in the thymus demonstrated an increasing or recovering trend. However, differences in MNC counts in the thymus between a royal jelly dose group and a group not administered royal jelly were not statistically significant (FIG. 8).

In addition, there were no differences in CD3-, B220-, CD4- and CD8-positive lymphocyte subset counts among these MNC in the liver and spleen (data not shown). On the other hand, during the time total MNC count in the thymus demonstrated an increasing trend in mice administered royal jelly, the counts of CD4⁺ and CD8⁺ cells as well as CD4⁺/CD8⁺ double positive cells were able to be observed to demonstrate a recovering trend in the proper balance of each (FIG. 9).

### CONCLUSION

Stress has become a part of daily life in modern society. In addition, stress has been verified to act deleteriously on immune function (23, 24), and increase susceptibility to illness (1-3). For this reason, methods for alleviating stress are essential for maintaining health, including somatic, mental and physiological aspects. Although royal jelly has been reported to be effective against oxidative stress in experiments using rats (25), there have yet to be any reports describing the activating effects of royal jelly on immune function in mice subjected to restraint stress, which is one of the models used to simulate mental stress. Since royal jelly has been reported to activate immune function through enhancement of antibody production and stimulation of proliferation of immunocompetent cells (11, 12, 16), an experiment was conducted in this study on the immunoregulatory function of the brain on immune modulation induced by restraint stress. In this experiment, a study was conducted as to the manner in which royal jelly demonstrates ameliorative effects on a serious immunodeficient state induced in mice subjected to restraint stress.

When stress was subject to mice by chronically restrain the mice for 12 hours two days a week over the course of 6 weeks, remarkable decreases were observed in peripheral leukocyte counts and MNC counts in the thymus, spleen and liver. In addition, remarkable decreases in lymphocytes coupled with remarkable increases in granular leukocytes were observed in the peripheral blood. When royal jelly was administered to these mice, the decreased lymphocyte counts increased and recovered to a considerable degree, while conversely, increased granular leukocyte counts were observed to decrease, thus confirming that lymphocytopenia and granulocytosis caused by restraint stress recover remarkably following administration of royal jelly. Royal jelly was presumed to contain a proliferation activating substance that acts on lymphocytes and an inhibitory factor that acts on granulocytes.

This finding that royal jelly up-regulates production of peripheral lymphocytes in mice under restraint stress coincides with a previous report stating that royal jelly contains a component that enhances proliferation of immunocompetent cells in mice (15). In addition, the result that royal jelly down-regulates stress-induced granulocytosis can be explained by several results indicating that royal jelly demonstrates an anti-inflammatory response mediated by inhibition of macrophage proinflammatory cytokines (26-28). These results are suggested to indicate that royal jelly demonstrates a significant, although incomplete, normalizing effect on circulatory lymphocytes and granular leukocytes, and that administration of royal jelly demonstrates an effective action on modulation of immune function inducted by restraint stress.

On the other hand, an explanation for being able to consider that royal jelly maintained homeostasis of circulatory immune cells under restraint stress is that a sustained supply of T cells from the thymus compensated for the loss of lymphocytes. This approach may coincide with the data from this study indicating that royal jelly partially restored the total number of cells in the thymus and the number of CD4- and CD8-positive T cells under stress. The additional supply of CD4 and CD8 T cells from the thymus along with B cells from bone marrow may have assisted in maintaining normal levels of peripheral T and B lymphocytes counts. This coincides with the result that royal jelly protects mice from impairment of hematopoietic function by inducing macrophage activity and proliferation of hematopoietic stem cells with respect to the hematopoietic function of X-ray irradiated mice (29,30).

The importance of royal jelly in up-regulation of thymus T cells supplying T cells to the peripheral blood and the induction of a systemic immune response has also been reported (24). The effect of royal jelly of improving changes in the number of MNC and subsets thereof in the liver and spleen were unable to be confirmed in this study royal jelly demonstrating this effect in the peripheral blood. In the experiment described here, although the effects of administration of royal jelly on the liver and spleen were observed in mice on day 42, the regulatory effect of royal jelly on immune cells of peripheral blood were already no longer observed at this time. Consequently, in the case of examining on day 14 or day 28 when this effect was observed in the peripheral blood, the possibility of observing the effects of royal jelly in these organs that are similar to those of the peripheral blood cannot be ruled out.

On the other hand, although differences were not statistically significant, thymus cells, for which counts were significantly reduced under restraint stress, were indicated to demonstrate favorable restorative effects attributable to administration of royal jelly for total cell counts and each of the subsets of CD4/CD8-positive cells as well as CD4-positive and CD8-positive T cells. When this is considered together with the recovery of T lymphocyte counts on days 14 and 28 along with the aforementioned effect in the thymus on day 42, royal jelly is presumed to have been intimately involved in differentiation of T cells in the thymus between days 14 and 28.

Another reason for the possible cause of royal jelly not demonstrating adequate restorative effects on MNC counts in the liver and spleen may be related to problems with migration and homing of restored lymphocytes in the thymus. Since lymphocyte migration and homing may have been impaired in the liver and spleen when subjected to stress, there is the possibility that this impairment of homing may have prevented the restorative effects of royal jelly on lymphocytes from being reflected in the number of lymphocytes in these organs. Further studies will be required to be conducted in the future in order to verify these presumptions.

This experiment, in which royal jelly ameliorated stress-induced lymphocytopenia and inhibited granulocytosis is unique in the sense that the royal jelly caused a proinflammatory response and an anti-inflammatory response simultaneously. Although royal jelly has been previously reported to induce a proinflammatory response while also being involved in the mechanism of an anti-inflammatory response, there have been no reports verifying the simultaneous occurrence of these phenomena in a single experimental system.

The finding that royal jelly simultaneously induced offsetting phenomena consisting of a lymphocyte-based proinflammatory response and an anti-inflammatory response is more likely to be the result of royal jelly having demonstrated a homeostatic function in terms of maintaining a harmonious relationship throughout the immune system rather than having been involved in these reactions separately. In any case, decreases in lymphocytes are known to cause a decrease in immunocompetence, and an excessive granulocytic response is known to be involved in certain types of illnesses as well as systemic inflammatory responses and tissue damage under stressful conditions. The finding that royal jelly inhibited stress-induced granulocytosis in this experiment suggests that royal jelly is effective for treatment of inflammatory responses and tissue damage caused by granular leukocytes. When considering on the basis of previously reported experiments and the findings reported here, the partial alleviation of the effects of restraint stress by royal jelly can be hypothesized to be the result of the various beneficial components of royal jelly having led to a favorable balance of effects for the host.

### REFERENCE DOCUMENTS

1. Sagiyama K, Tsuchida M, Kawamura H, Wang S, Li C, Bai X, Nagura T, Nozoe S, AboT (2004) Age-related bias in function of natural killer T cells and gramlocytes after stress: reciprocal association of steroid homones anf sympathetic nerves. Clin Exp Immunol 135:56-63
2. Pruett S B, Fan R, Myers L P, Wu W J, Collier S (2000) Quantitative analysis of theneuroendocrine-immune axis: linear modeling of the effects of exogenous corticosterone and restraint stresson lymphocyte subpopulations in the spleen and thymus in female B6C3F1 mice. Brain Behav Immun 14:270-87
3. Breslau N, Davis G C (1986) Chronic stress and major depression. Arch Gen Psychiatry 43:309-14
4. Galosy R A, Clark L K, Vasko M R, Crawford I L (1981) Neurophysiology and neurophafmacology ofcardiovascular regulation and stress. Neusosci Biobehav Rev 5:137-75
5. Tapp W N, Natelson B H (1988) Consequences of stress: a multiplicative function of health status. FASEBJ 2:2268-71
6. Millan S, Gonzalez-Quizano M I, Giordano M, Soto L, Martin A I, Lopez-Calderon A (1996) Short and longrestraint differentially affect humoral and cellular immune functions. Life Sci 59:1431-42
7. Fujiwara R, Shibata H, Komori T, Ybkoyama M M, Okazaki Y, Ohmori M (1999) The mechanisms of immune suppression by high pressure stress in mice. J Pharm Pharmacol 51:1397-404
8. Shimzu T, Kawamura T, Miyaji C, Oya H, Bannai M, Yamamoto S, Weerasinghe A, Halder R C, WatanabeH, Hatakeyama K, Abo T (2000) Resistance of extrathymic T cells to stress and the role of endogenousglucocolticoids in stress associated immunosuppression. Scand J Immunol 51:285-92
9. Tarcic N, Ovadia H, Weiss D W, Weidenfeld J (1998) Restraint stress-induced thymic involution and cell apoptosis are dependent on endogenous glucocorticoids. J Neuroimmunol 82:40-6
10. Ayala A, Herdon C D, Lehman D L, DeMaso C M, Ayala C A, Chaudry I H (1995) The induction of accelerated thymic programmed cell death during polymicrobial sepsis: control by corticosteroids but nottmmor necrosis factor. Shock 3:259-67
11. Sver L, Orsolic N, Tadic Z, Njari B, Valpotic I, Basic I (1996) A royal jery as a new potentialimmmmomodulator in rats and mice. Comp Immunol Microbiol Infect Dis 19:31-8
12. Okamoto I, Taniguchi Y, Kunikata T, Kohno K, Iwaki K, Ikeda M, Kurmoto M (2003) Mjor royal jellyproten modulates immune responses in vitro and in vivo. Life Sci 73:2029-45
13. Nagai T, Inoue R, Suzuki N, Nagashima T (2006) Antioxidant properties of enzymatic hydrolysates fromroyal jelly. J Med Food 9:363-7
14. Narita Y, Nomura J, Ohta S, Inoh Y, Suzuki K M, Araki Y, Okada S, Matsumoto I, Isohama Y, Abe K, Miyata T, Mishima S (2006) Royal jelly stimulates bone formation: physiologic and nutrigenomic studies with mice and cell lines. Biosci Biotechnol Biochem 70:2508-14
15. Hidaka S, Okamoto Y, Uchiyama S, Nakatsmma A, Hashikinoto K, Ohnishi S T, Yamaguchi M (2006) Royaljelly prevents osteoporosis in rats: beneficial effects in ovariectomy model and in bone tissue culture model. Evid Based Complement Alternat Med 3:339-48
16. Majtan J, Kovacova E, Bilikova K, Simuth J (2006) The immunostimulatory effect of the recombinant apalbumin 1-major honeybee royal jelly protein-on TNF alpha release. Int Immunopharmacol 6:269-78
17. Stocker A, Schramel P, Kettrup A, Bengsch E (2005) Trace and mineral elements in royal jelly and homeostatic effects. J Trace Elem Med Biol 19:183-9
18. Yamaguchi K. Innovation of natural beekeeping and quality control of beeproducts for promisingapiculture. Current problems of beekeeping and recurrence of natural bee-culture for resolution of the problems. J Yunan Agricultural Univ 2004; 19:322-9.
19. Moroda T, Iiai T, Tsukahara A, Fukmda M, Suzuki S, Tada T, Hatakeyama K, Abo T (1997) Association of granulocytes with ulcer formation in the stomach of rodents exposed to restraint stress. Biomed Res 18:423-37
20. Ito C, Shen H, Toyota H, Kubota Y, Sakurai E, Watanabe T, Sato M (1999) Effects of the acute and chronic restraint stresses on the central histaminergic neuron system of Fisher rat. Neurosci Lett 262:143-5
21. Tilbrook A J, Canny J B, Serapaglia M D, Ambrose T J, Clarke I J (1999) Suppression of the secretion of luteinizeing hormone due to isolation/restraint stress in gonadectomised rams and wews is influenced by sex steroids. J Endocrinol 160:469-81
22. Mannoor M K, Weerasinghe A, Halder R C, Morshed S R M, Ariyasjnghe A, Watanabe H, Sekikawa H, Abo T (2001) Resistance to malarial infection is achieved by the cooperation of NK1.1+ and NK1.1− subsets of intermediate TCT cells which are constituents of innate immunity. Cell Immunol 211:96-104
23. Carrasco G. A, Van de Kar L. D (2003) Neuroendocrine pharmacology of stress, Eur J Pharmacol 463(1-3) 235-272.
24. Chrousos G P (2000) The stress response and immune function: clinical implications. The 19999 Novera H. Spector Lecture, Ann N Y Acad Sci 917:38-66.
25. El-Nekeety A A, El-Kholy W, Abbas N F, Ebaid A, Amra H A, Abdel-Wahhab M A (2007) Efficacy of royal jelly against the oxidative stress of fumonisin in rats. Toxicon. 50(2)256-69
26. Kohno K, Okamoto I, Sano O, Arai N, Iwaki K, Ikeda M, Kurimoto M (2004) Royal jelly inhibits the production of proinflammatory cytokines by activated macrophages. Biosci Biotechnol Biochem 68:138-45
27. Emori Y, Oka H, Kobayashi N, Ohya O, Tamaki H, Hayashi Y, Nomoto K (1999) Protective effect of royal jelly on immune dysfunction in aged mice. Biotherapy 13:281-7
28. Fujii A, Kobayashi S, Kuboyama N, Furukawa Y, Kaneko Y, Ishihara S, Yamamoto H, Tamura T (1990) Augmentation of wound healing by royal jelly in streptozotocin-diabetic rats. Jpn J Phamaco 53:331-7
29. 2006 Cheng Y H, Ding S T, Chang M H (2006). Effect of fumonisins on macropharge immune functions and gene expression of cytokins in broilers, Arch. Anim. Nutr. 60(4):267-276.
30. Emori Y, H., Oka H, Ohya O, Tamaki H, Hayashi H, Nomoto K (1998). The protective effect of royal jelly against the hemopoiesis dysfunction in X-irradiated mice, Biotherapy 12:313-319.

1. An agent for ameliorating stress-induced immune function modulation which comprises royal jelly.

2. A pharmaceutical having an ameliorative action on stress-induced immune function modulation, which comprises royal jelly as an active ingredient thereof.

3. A food or beverage having an ameliorative action on stress-induced immune function modulation, which comprises royal jelly as an active ingredient thereof.

4. A food additive having an ameliorative action on stress-induced immune function modulation, which comprises royal jelly as an active ingredient thereof.

5. A method for ameliorating stress-induced immune function modulation which comprises ingesting royal jelly.
